(19) United States
(12) Patent Application Publication
(10) Pub. No.: US 2006/0189699 A1
(43) Pub. Date: Aug. 24, 2006

(54) Antimicrobial trinervitane derivatives

(75) Inventors: Rodney Warren Rickards, Weetangera (AU); Chunjiu Zhao, Changchun (AU); Stephen Charles Trowell, Campbell (AU)

Correspondence Address: GREENLEE WINNER AND SULLIVAN P C, 4875 PEARL EAST CIRCLE, SUITE 200, BOULDER, CO, 80301, US

(73) Assignee: Commonwealth Scientific and Industrial Researh Org, Campbell (AU)

(21) Appl. No.: 10/544,174

(22) PCT Filed: Feb. 3, 2004
(86) PCT No.: PCT/AU04/00123

§ 371 Date: Mar. 1, 2006

(30) Foreign Application Priority Data
Feb. 4, 2003 (AU) 2003900555

(51) Int. Cl. A61K 31/045; C07C 35/22

(52) U.S. Cl. 514/729; 568/817

(57) ABSTRACT

The present invention relates to a novel class of antimicrobial compounds. These trinervitane compounds can be used for a variety of purposes such as for the treatment of microbial infections and diseases, and as disinfectant agents.

### FIELD OF THE INVENTION

The present invention relates to a novel class of antimicrobial compounds. These compounds can be used for a variety of purposes such as for the treatment of microbial infections and diseases, and as disinfectant agents.

### BACKGROUND OF THE INVENTION

Antibiotics, compounds with selective toxicity against infectious microorganisms, present humanity with enormous benefits and are credited with saving many millions of lives since their introduction in the 20th century. Today there is a continuing need for new antibiotics to assist in the management of multiply resistant pathogens (e.g. multiply resistant Staphyloccus aureus or vancomycin-resistant Enterococcus) or to provide improved therapies for difficult-to-treat pathogens such as Mycobacterium tuberculosis, the causative agent of tuberculosis. Selectively toxic compounds also have utility as veterinary antibiotics and growth enhancers, where there is a need to develop agents with different modes of action from those used in humans, and also as preservatives and antisepsis agents in a wide range of medical and industrial processes and products.

Insects and terrestrial invertebrates face infection by many opportunistic microbial pathogens, yet they are a successful group of organisms which have been present on earth for hundreds of millions of years and are today represented by many millions of species, far more than any other group of macroorganisms. Insects and other terrestrial invertebrates must therefore have efficient methods for avoiding or overcoming potential infections.

Insects share with mammals and other organisms an “innate” immune system based on non-specific phagocytosis of foreign material by haemocytes, and production of a range of antimicrobial peptides such as defensins, cecropins and attacins in response to general microbial inducers such as lipopolysaccharide and (1,3)-beta-D-glucans. However, there has been no evidence from insects, or any other invertebrate, for the presence of a clonal, inducible-immune system of the B-lymphocyte/T-lymphocyte type that typifies mammalian responses to infection. Insects may therefore have other, undiscovered, defensive systems to protect themselves against microbial invasion.

There has been little previous evidence for the synthesis of non-peptide antibiotics by insects. A survey of 102 species of North American arthropods in the 1950's [1] revealed only two active extracts, and these were presumed to be active due to the presence of quinones, reactive compounds of no value as antibiotics. An antibacterial compound, p-hydroxycinnamaldehyde, has recently been isolated from a Korean sawfly [2], however no data on the mammalian toxicity of this compound was presented.

Between 1997 and 1999, the present Applicants assembled a large collection of terrestrial invertebrates from the east coast of Australia and extracted a number of them and screened the extracts for biological activity. As a result the present Applicants identified antibiotic trinervitadienes from a species of nasute termite, Nasutitermes triodiae (WO 01/90035).

Since microbial resistance to known antibiotics is increasing, there is a need for farther compounds which can be used as antimicrobial agents.

### DISCLOSURE OF THE INVENTION

The present inventors have identified new trinervitanes which possess antimicrobial activity.

In one aspect, the present invention provides a compound of the formula:

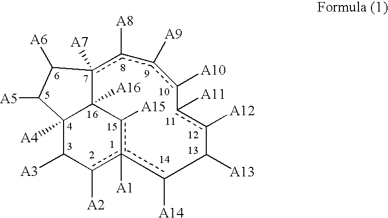

wherein the compound comprises at least one double bond between the carbon atoms selected from the group consisting of: C1 and C14, C9 and C10, and C7 and C8; and

wherein; each

[symbol]

independently denotes a single or double bond or an epoxidised bond, and

(i) substituents A¹ to A¹⁶ are selected, independently, from H, OH, O, SH, NH₂, lower alkyl, lower alkene, lower alkyne, lower alkoxy, lower carboxy, lower aldehyde groups, lower ketone groups, lower ester groups, lower acyloxy groups, lower alcohol groups, lower alkylthio, lower alkylamino, lower alkysulfonyl, lower alkysulfinyl and lower alkylsulfonyloxy, or

(ii) any one or more of substituent pairs A¹ and A², A¹ and A³, A² and A³, A² and A⁴, A³ and A⁴, A³ and A⁵, A⁴ and A⁵, A⁴ and A⁶, A⁵ and A⁶, A⁵ and A⁷, A⁶ and A⁷, A⁶ and A⁸, A⁷ and A⁸, A⁷ and A⁹, A⁸ and A⁹, A⁸ and A¹⁰, A⁹ and A¹⁰, A⁹ and A¹¹, A¹⁰ and A¹¹, A¹⁰ and A¹², A¹¹ and A¹², A¹¹ and A¹³, A¹² and A¹³, A¹² and A¹⁴, A¹³ and A¹⁴, A¹ and A¹³, A¹ and A¹⁴, and A² and A¹⁴ form a substituted or unsubstituted heterocyclic group, wherein any substituents, including A¹⁵ and A¹⁶, not forming a substituted or unsubstituted heterocyclic ring, are selected independently from H, OH, O, SH, NH₂, lower alkyl, lower alkene, lower alkyne, lower alkoxy, lower carboxy, lower aldehyde groups, lower ketone groups, lower ester groups, lower acyloxy groups, lower alcohol groups, lower alkylthio, lower alkylamino, lower alkysulfonyl, lower alkysulfinyl and lower alkylsulfonyloxy; and

wherein A¹ or A⁷ may be absent; and

wherein any one of A², A³, A⁵, A⁶ and A⁸ to A¹⁵ may be bonded to the ring structure of Formula (1) by a single or double bond or an epoxidised bond; and

optical isomers, hydrates, solvates and/or salts or derivates salts thereof;

with the proviso that the compound is not 7(8),11(12),15(17)-trinervitatriene-2α,3α-diol.

In a preferred embodiment, the compound comprises at least one double bond between the carbon atoms selected from the group consisting of: C1 and C14, and C9 and C10.

In a further preferred embodiment the compound of Formula (1) has three, four or more double bonds. More preferably, the compound comprises double bonds at least between the carbon atoms selected from the groups consisting of;

i) C1 and C14, C9 and C10, and C8 and A⁸, ii) C1 and C14, C15 and A¹⁵, and C8 and A⁸, or iii) C1 and C14, C15 and A¹⁵, and C7 and C8.

Even more preferably, the compound comprises a double bond at least between C1 and C14.

Preferably, A¹, A², A³, A⁵, A⁶, A⁷, A⁹, A¹⁰, A¹¹, A¹³, A¹⁴ and A¹⁶ are selected, independently, from H, OH, O, SH, NH₂ and OR More preferably, A¹, A², A³, A⁵, A⁶, A⁷, A⁹, A¹⁰, A¹¹, A¹³, A¹⁴ and A¹⁶ are selected, independently, from H, OH and OR, R in the group OR is a lower alkyl as defined herein (preferably, methyl or ethyl) or lower acyl. Even more preferably, A⁷ and/or A¹⁶ are H.

Preferably, A⁴ and A¹² are selected, independently, from lower alkyl, lower carboxy, lower aldehyde groups, lower ketone groups, lower ester groups, lower acyloxy and lower alcohol groups. More preferably, A⁴ and A¹² are selected, independently, from methyl, hydroxymethyl, formyl and carboxyl. Even more preferably, A⁴ and A¹² are methyl.

Preferably, A⁸ and A¹⁵ are selected from lower alkyl, lower alkene or lower alkyne. More preferably, A⁸ and A¹⁵ are selected from methyl and CH₂. Most preferably, A⁸ and A¹⁵ are CH₂.

It is also preferred that at least two of said A¹ to A¹⁶ are OH or OR groups, wherein R is as defined above. More preferably, at least A² and A³ are OH.

In another preferred embodiment, the compound comprises the formula:

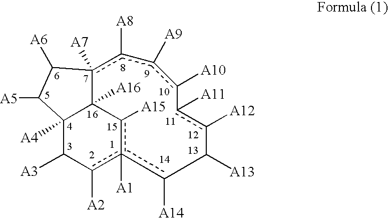

wherein the compound comprises at least one double bond between the carbon atoms selected from the group consisting of: C1 and C14, C9 and C10, and C7 and C8; and

wherein; each

[symbol]

independently denotes a single or double bond or an epoxidised bond, and

wherein substituents A¹ to A¹⁶ are selected, independently, from H, OH, O, SH, NH₂, lower alkyl, lower alkene, lower alkyne, lower alkoxy, lower carboxy, lower aldehyde groups, lower ketone groups, lower ester groups, lower acyloxy groups, lower alcohol groups, lower alkylthio, lower alkylamino, lower alkysulfonyl, lower alkysulfinyl and lower alkylsulfonyloxy, and

wherein A¹ or A⁷ may be absent; and

wherein any one of A², A³, A⁵, A⁶ and A⁸ to A¹⁵ may be bonded to the ring structure of Formula (1) by a single or double bond or an epoxidised bond; and

optical isomers, hydrates, solvates and/or salts or derivatives thereof;

with the proviso that the compound is not 7(8),11(12),15(17)-trinervitatriene-2α,3α-diol.

In another preferred embodiment, the compound comprises the formula:

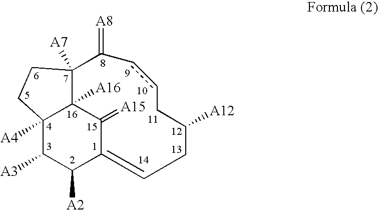

wherein;

[symbol]

denotes a single or double bond; and

wherein substituents A², A³, A⁴, A⁷, A⁸, A¹², A¹⁵ and A¹⁶ are as defined above in elation to Formula (1), and

optical isomers, hydrates, solvates and/or salts or derivatives thereof.

In another preferred embodiment, the compound comprises the formula:

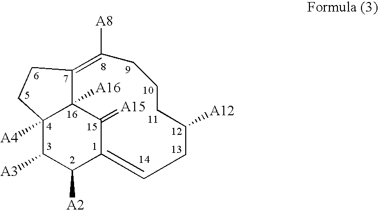

wherein substituents A², A³, A⁴, A⁸, A¹², A¹⁵ and A¹⁶ are as defined above in relation to Formula (1), and

optical isomers, hydrates, solvates and/or salts or derivatives thereof.

More preferably, the compound is selected from the group consisting of: (9E)-1(14),8(19),9(10),15(17)-trinervitatatriene-2β,3α-diol (P); 1(14),8(19),15(17)-trinervitatriene-2β,3α-diol (Q); and 1(14),7(8),15(17)-trinervitatriene-2β,3α-diol (R).

Preferably, the compound is in a substantially purified form. The compounds of the present invention can be synthesized by known chemical procedures. In some cases the compounds may be substantially purified from a termite of the genus Nasutitermes. Even more preferably, the compound is substantially purified from soldier caste of Nasutitermes exitiosus. In other cases, compounds of the invention are obtained by performing standard chemical reactions on compounds obtained from natural sources, e.g. from termites, and then functionalized to arrive at the desired compound.

Preferably, the salts are pharmaceutically/veterinary-acceptable salts. Suitable pharmaceutically/veterinary-acceptable salts of the compound of Formula (1) include non-toxic salts such as acid addition salts such as an inorganic acid addition salt (e.g. hydrochloride, sulfate, phosphate, etc.), an organic acid addition salt (e.g. formate, acetate, trifluoroacetate, etc.), a salt with an amino acid (e.g. arginine salt, etc.), a metal salt such as an alkali metal salt (e.g. sodium salt, potassium salt, etc.) and an alkaline earth metal salt (e.g. calcium salt, magnesium salt, etc.), an ammonium salt, an organic base addition salt (e.g. trimethylamine salt, triethylamine salt, etc.) and the like.

The compounds of the present invention also includes various crystal forms.

The compounds of the present invention also include isomers of those represented herein by specific Formulae. These isomers include optical isomers, stereoisomers and geometric isomers.

In another aspect, the present invention provides a pharmaceutical and/or veterinary formulation for treating a microbial infection or disease in a subject, said formulation comprising a compound according to any of the Formula (1) to (3) in admixture with a suitable pharmaceutically/veterinary-acceptable excipient.

In a further aspect, the present invention provides a composition comprising a compound according to the invention in admixture with at least one diluent.

The compounds of the present invention have antimicrobial activity and are therefore useful as, for example, human or veterinary or aquatic antibiotics, as antiseptic/disinfectant agents in industrial or other processes, as agricultural chemicals, or as food preservatives.

Thus, in a further aspect the present invention provides a method for treating or preventing a microbial infection or disease in a subject, the method comprising administering to the subject an effective amount of a compound having the formula:

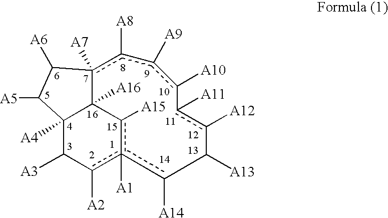

wherein the compound comprises at least one double bond between the carbon atoms selected from the group consisting of: C1 and C14, C9 and C10, and C7 and C8; and

wherein; each

[symbol]

independently denotes a single or double bond or an epoxidised bond, and

(i) substituents A¹ to A¹⁶ are selected, independently, from H, OH, O, SH, NH₂, lower alkyl, lower alkene, lower alkyne, lower alkoxy, lower carboxy, lower aldehyde groups, lower ketone groups, lower ester groups, lower acyloxy groups, lower alcohol groups, lower alkylthio, lower alkylamino, lower alkysulfonyl, lower alkysulfinyl and lower alkylsulfonyloxy, or

(ii) any one or more of substituent pairs A¹ and A², A¹ and A³, A² and A³, A² and A⁴, A³ and A⁴, A³ and A⁵, A⁴ and A⁵, A⁴ and A⁶, A⁵ and A⁶, A⁵ and A⁷, A⁶ and A⁷, A⁶ and A⁸, A⁷ and A⁸, A⁷ and A⁹, A⁸ and A⁹, A⁸ and A¹⁰, A⁹ and A¹⁰, A⁹ and A¹¹, A¹⁰ and A¹¹, A¹⁰ and A¹², A¹¹ and A¹², A¹¹ and A¹³, A¹² and A¹³, A¹² and A¹⁴, A¹³ and A¹⁴, A¹ and A¹³, A¹ and A¹⁴, and A² and A¹⁴ form a substituted or unsubstituted heterocyclic group, wherein any substituents, including A¹⁵ and A¹⁶, not forming a substituted or unsubstituted heterocyclic ring, are selected independently from H, OH, O, SH, NH₂; lower alkyl, lower alkene, lower alkyne, lower alkoxy, lower carboxy, lower aldehyde groups, lower ketone groups, lower ester groups, lower acyloxy groups, lower alcohol groups, lower alkylthio, lower alkylamino, lower alkysulfonyl, lower alkysulfinyl and lower alkylsulfonyloxy; and

wherein A¹ or A⁷ may be absent; and

wherein any one of A², A³, A⁵, A⁶ and A⁸ to A¹⁵ may be bonded to the ring structure of Formula (1) by a single or double bond or an epoxidised bond; and

optical isomers, hydrates, solvates and/or salts or derivatives thereof.

Also provided is the use of a compound of the invention for the manufacture of a medicament for treating or preventing a microbial infection or disease in an subject.

Furthermore, in another aspect the present invention provides a method for disinfecting a surface (e.g. a hard surface such as kitchen bench tops, bathroom tiles and the like) or a solution, said method comprising exposing said surface or solution to an effective amount of a compound having the formula:

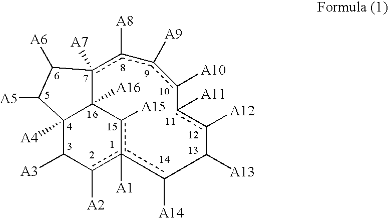

wherein the compound comprises at least one double bond between the carbon atoms selected from the group consisting of: C1 and C14, C9 and C10, and C7 and C8; and

wherein; each

[symbol]

independently denotes a single or double bond or an epoxidised bond, and

(i) substituents A¹ to A¹⁶ are selected, independently, from H, OH, O, SH, NH₂, lower alkyl, lower alkene, lower alkyne, lower alkoxy, lower carboxy, lower aldehyde groups, lower ketone groups, lower ester groups, lower acyloxy groups, lower alcohol groups, lower alkylthio, lower alkylamino, lower alkysulfonyl, lower alkysulfinyl and lower alkylsulfonyloxy, or

(ii) any one or more of substituent pairs A¹ and A², A¹ and A³, A² and A³, A² and A⁴, A³ and A⁴, A³ and A⁵, A⁴ and A⁵, A⁴ and A⁶, A⁵ and A⁶, A⁵ and A⁷, A⁶ and A⁷, A⁶ and A⁸, A⁷ and A⁸, A⁷ and A⁹, A⁸ and A⁹, A⁸ and A¹⁰, A⁹ and A¹⁰, A⁹ and A¹¹, A¹⁰ and A¹¹, A¹⁰ and A¹², A¹¹ and A¹², A¹¹ and A¹³, A¹² and A¹³, A¹² and A¹⁴, A¹³ and A¹⁴, A¹ and A¹³, A¹ and A¹⁴, and A² and A¹⁴ form a substituted or unsubstituted heterocyclic group, wherein any substituents, including A¹⁵ and A¹⁶, not forming a substituted or unsubstituted heterocyclic ring, are selected independently from H, OH, O, SH, NH₂, lower alkyl, lower alkene, lower alkyne, lower alkoxy, lower carboxy, lower aldehyde groups, lower ketone groups, lower ester groups, lower acyloxy groups, lower alcohol groups, lower alkylthio, lower alkylamino, lower alkysulfonyl, lower alkysulfinyl and lower alkylsulfonyloxy; and

wherein A¹ or A⁷ may be absent; and

wherein any one of A², A³, A⁵, A⁶ and A⁸ to A¹⁵ may be bonded to the ring structure of Formula (1) by a single or double bond or an epoxidised bond; and

optical isomers, hydrates, solvates and/or salts or derivatives thereof

The antimicrobial compound of the present invention may be effective against bacteria, both gram-positive and gram-negative, as well as fungi and protozoans.

Also provided is a kit comprising a compound of the invention. The kit preferably further comprises a diluent which is either in admixture with the compound or can be in a separate vial and hence can be combined with the compound before use.

Preferably, the compound is provided in a composition which is suitable to be applied or administered to an animal or plant.

In a further embodiment, the kit comprises other antimicrobial compounds such as those described in WO 01/90035.

Preferably, the kit further comprises information and/or instructions for use of the kit.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting examples and with reference to the accompanying figures.

### BRIEF DESCRIPTION OF THE ACCOMPANYING FIGURES

FIG. 1: Reverse-phase HPLC profile of the antimicrobial extract prepared from N. exitiosus soldiers.

FIG. 2: Reverse-phase HPLC profile of the inactive extract prepared from N. exitiosus workers.

FIG. 3: Purity of compound P by reverse-phase HPLC.

FIG. 4: Proton NMR spectrum of compound P (in CDCl₃).

FIG. 5: APT spectrum of compound P (in CDCl₃).

FIG. 6: COSY spectrum of compound P (in CDCl₃).

FIG. 7: HMQC spectrum of compound P (in CDCl₃).

FIG. 8: HMBC spectrum of compound P (in CDCl₃).

FIG. 9: NOESY spectrum of compound P (in CDCl₃).

### DETAILED DESCRIPTION OF THE INVENTION

Definitions

The term “lower” is intended to mean a group having 1, 2, 3, 4, 5 or 6 carbon atom(s), unless otherwise provided.

Suitable “lower alkyl” and lower alkyl moieties in the terms “lower alkoxy”, “lower alkythio”, “lower alkylamino”, “lower alkylsulfonyl”, “lower alkylsulfinyl” and “lower alkylsulfonyloxy” may be straight or branched such as methyl, ethyl, propyl, isopropyl, butyl, isobutyl, tert-butyl, pentyl, hexyl or the like.

Suitable “lower alkene” groups include, but are not limited to, CH₂, CHCH₃, CHCH₂, CHCHCH₃ and the like. Furthermore, suitable “lower alkyne” groups include, but are not limited to, CCH, CCCH₃ and the like.

Suitable “lower alkoxy” groups include, but are not limited to, methoxy, ethoxy, propoxy, isopropoxy, butoxy, isobutoxy, tert-butoxy and the like.

Suitable “lower carboxy” groups include, but are not limited to, carboxy, carboxymethyl, carboxyethyl, carboxypropyl, carboxyisopropyl, carboxybutyl, carboxyisobutyl, carboxy tert-butyl and the like.

Suitable “lower aldehyde” groups include, but are not limited to, those selected from aldehyde groups such as methanal, ethanal, propanal, isopropanal, butanal, isobutanal, tert-butanal and the like.

Suitable “lower ketone” groups include, but are not limited to, those selected from ketone groups such as ethanone, propanone and the like.

Suitable “lower ester” groups include, but are not limited to, methanoate, ethanoate, propanoate, isopropanoate, butanoate, isobutanoate, tert-butanoate and the like.

Suitable “lower acyloxy” groups include, but are not limited to, acetoxy, propionyloxy, butyryloxy and the like.

Suitable “lower alcohol” groups include, but are not limited to, methanol, ethanol, propanol, isopropanol, butanol, isobutanol, tert-butanol and the like.

Suitable “lower alkylthio” groups include, but are not limited to, methylthio, ethylthio, propylthio, butylthio and the like, and lower alkyl thio substituted lower alkyl such as methylthiomethyl, methylthioethyl, methylthiopropyl, methylthiobutyl, ethylthiomethyl, ethylthioethyl, ethylthiopropyl, ethylthiobutyl and the like.

Suitable “lower alkylamino” groups include, but are not limited to, methylamino, ethylamino, propylamino, butylamino and the like, and mono or di(lower alkyl) amino substituted lower alkyl such as methylaminomethyl, methylaminoethyl, methylaminopropyl, methylaminobutyl, ethylaminomethyl, ethylaminoethyl, ethylaminopropyl, ethylaminobutyl, dimethylaminomethyl, dimethylaminoethyl, dimethylaminopropyl, dimethylaminobutyl, diethylaminomethyl, diethylaminoethyl, diethylaminopropyl, diethylaminobutyl and the like.

Suitable “lower alkylsulfonyl” groups include, but are not limited to, methylsulfonyl, ethylsulfonyl, propylsulfonyl, butylsulfonyl and the like.

Suitable “lower alkylsulfinyl” groups include, but are not limited to, methylsulfinyl, ethylsulfinyl, propylsulfinyl, butylsulfinyl and the like.

Suitable “lower alkylsulfonyloxy” groups include, but are not limited to, methylsulfonyloxy, ethylsulfonyloxy, propylsulfonyloxy, butylsulfonyloxy and the like.

Suitable substituted or unsubstituted heterocyclic groups include, but are not limited to, groups having a carbon and oxygen backbone of 5 to 8 atoms (inclusive of the 2-3 carbon atoms contributed by the Formula (1) structure), including cyclic acetals and cyclic carbonates. Such heterocyclic groups may be substituted by one or more of OH, O, SH, NH₂, lower alkyl, lower alkene, lower alkyne, lower alkoxy, lower carboxy, lower aldehyde groups, lower ketone groups, lower ester groups, lower acyloxy, lower alcohol groups, lower alkylthio, lower alkylamino, lower alkysulfonyl, lower alkysulfinyl and lower alkylsulfonyloxy.

The general Formulae (1, 2 and 3) provided herein depict the carbon atoms forming the ring structure as carbons I to 16. As defined herein the compounds represented by the general Formulae may comprise further carbon atoms as part of substituents A¹ to A¹⁶. With regard to specific compounds and “carbon skeletons” provided herein (for example, compounds P, Q, R, S, T and U; and carbon skeletons V, W, X and Y), these further carbon atoms are specifically numbered. For example, a methyl group at position A⁸ in Formula (1) is represented as C19 in compound R. A similar carbon numbering system is used when referring to prior art molecules. For instance, as the skilled addressee would be aware, 7(8),11(12),15(17)-trinervitatriene-2α,3α-diol is generally represented in Formula (1) as 7(8),11(12),15(A¹⁵)-trinervitatriene-2α,3α-diol.

As used herein, the term “substantially purified” refers to a compound that has been separated from the lipids, nucleic acids, polypeptides, and other contaminating molecules with which it is associated in its native state. Preferably, the substantially purified compound is at least 60% free, preferably at least 75% free, and most preferably at least 90% free from other components with which it is naturally associated.

An “effective amount” of the compound is that amount necessary or sufficient to treat or prevent antimicrobial growth. The effective amount can vary depending on such factors as the size and weight of the subject, the type of illness, concentration of microbes, etc. One of ordinary skill in the art would be able to study the aforementioned factors and make the determination regarding the effective amount of the compound without undue experimentation.

The “subject” can be any living organism prone to microbial, especially bacterial, infection. Preferably the subject is a vertebrate or a plant. More preferably, the vertebrate is a fish, bird or a mammal. More preferably, the mammal is a human, a livestock animal (e.g. sheep, cow, horse, goat, etc) or a companion animal (e.g. cat, dog, etc).

Isolation, Derivatization and Synthesis

At least some of the compounds of the present invention are naturally occurring trinervitanes which have been obtained from native Australian termites, and are likely to be present in the same and possibly in related species in other countries. Access to larger quantities of these and related natural trinervitanes, which would be needed for therapeutic use, could be obtained from cultured colonies of the appropriate termites, or alternatively by laboratory synthesis of the desired compounds. Analogues of the natural materials, of the types described herein, could be obtained by chemical conversion from the natural materials, or alternatively by total synthesis in cases where such a route would be more efficaceous.

The synthesis of the basic tricyclic nucleus of the trinervitane diterpenes has been accomplished by means of Robinson annelation and McMurry coupling to yield oxygenated trinervitadiene products carrying olefinic functionality at 1(15),8(9)- or 1(15),8(19)-positions [21]. Furthermore, a trinervitatriene-2,3-diol carrying olefinic functionality at the 7(8),11(12),15(17)-positions has been synthesised by chemically simulating the proposed biogenetic route to such natural products [10, 22, 23]. Extension or adaptation of these routes using chemical reactions well known and described in the art [cf., for example, 28, 29], or the development of purpose designed synthetic routes again using chemical reactions well known and described in the art [cf., for example, 28, 29], would provide convenient access to the natural trinervitanes and their analogues.

Antimicrobial Compositions and the Use Thereof

The compounds of the present invention have antimicrobial activity and are therefore useful as, for example, human or veterinary or aquatic antibiotics, as antiseptic/disinfectant agents in industrial or other processes, as agricultural chemicals (including those compositions which can be applied to plants to control microbial infections), or as food preservatives. Further applications include, but are not limited to, inhibition of growth of microbial pathogens in environmental situations, reduction or prevention of microbial colonisation of medical media including washing solutions, ointments and the like.

Also provided is a method of treating/(disinfecting and cleaning) medical indwelling devices comprising administering a composition comprising an effective amount of a compound of the invention. These devices may advantageously include, for example, any indwelling device, for example catheters, orthopedic devices and implants.

The compounds according to the present invention can be used against a broad range of microorganisms causing various infectious diseases and are effective to prevent, alleviate or cure diseases caused by these pathogens.

Examples of bacteria or bacterium-like microorganisms on which the compounds of the invention are effective include, but are not limited to, bacilli such as Bacillus subtilis, Bacillus anthracis, Bacillus cereus; staphylococci such as Streptococcus pyogenes, Streptococcus haemolyticus, Streptococcus faecalis, Streptococcus pneumoniae, Staphylococcus aureus; peptostreptococci such as Neisseria gonorrhoeae, Escherichia coli, Citrobacter sp., Shigella sp., Klebsiella pneumoniae, Enterobacter sp., Serratia sp., Proteus sp., Pseudomonas aeruginosa, Haemophilus influenzae, Acinetobacter sp., Campylobacter sp., and Chlamydia sp. such as Chlamydia trachomatis and Chlamydia pneumoniae.

The methods of the invention may be used for the treatment or prevention of an microbial infection or disease selected from, for example, bacterial infection of wounds including surgical wounds, lung infections (e.g. tuberculosis), skin infections, and systemic bacterial infections. For instance, diseases which can be treated or prevented by the antimicrobial compounds of the present invention include, but are not limited to, anthrax, food poisoning, folliculitis, furuncle, carbuncle, erysipelas, phlegnon, lymphangitis/lymphadenitis, felon, subcutaneous abscess, spiradenitis, acne agminata, infectious atheroma, perianal abscess, masitadenitis, superficial secondary infections after trauma, burn or surgery trauma, pharyngolaryngitis, acute bronchitis, tonsillitis, chronic bronchitis, bronchiectasis, diffuse panbronchiolitis, secondary infections of chronic respiratory diseases, pneumonia, pyelonephritis, cystitis, prostatitis, epididymitis, gonococcal urethritis, non-gonococcal urethritis, cholecystitis, cholangitis, bacillary dysentery, enteritis, adnexitis, intrauterine infections, bartholinitis, blepharitis, hordeolum, dacryocystitis, tarsadenitis, keratohelcosis, otitis media, sinusitis, paradentosis, pericoronitis, gnathitis, peritonitis, endocarditis, septicemia, meningitis, and skin infections.

The compounds of the present invention are also effective on various microorganisms causing veterinary diseases, such as those belonging to the genera Escherichia, Salmonella, Pasteurella, Haemophilus, Bordetella, Staphylococcus, and Mycoplasma. Illustrative examples of the veterinary diseases include those of fowl, such as colibacillosis, pullorum disease, avian paratyphosis, fowl cholera, infectious coryza, staphylomycosis, and mycoplasmosis; those of pigs, such as colibacillosis, salmonellosis, pasteurellosis, hemophilus infections, atrophic rhinitis, exudative epidermitis, and mycoplasmosis; those of cattle, such as colibacilosis, salmonellosis, hemorrhagic septicemia, mycoplasmosis, bovine contagious pleuropneumonia, and bovine mastitis; those of dogs, such as colisepsis, salmonellosis, hemorrhagic septicemia, pyometra, and cystitis; those of cats, such as exudative pleurisy, cystitis, chronic rhinitis, and hemophilus infections; and those of kittens, such as bacterial diarrhea and mycoplasmosis.

The invention also provides a method for treatment and/or prophylaxis of parasitic infections, particularly those caused by protozoan parasites. Included among the protozoan parasites are those of the genera Giardia, Trichomonas, Leishmania, Trypanosoma, Crithidia, Herpetomonas, Leptomonas, Histomonas, Eimeria, Isopora and Plasmodium. An example of a parasitic infection caused by Plasmodiuyn is malaria

The invention also provides a method for treatment and/or prophylaxis of fungal infections. Such fungal infections (mycoses) may be, for example, cutaneous, subcutaneous or systemic. Superficial mycoses include tinea capitis, tinea corporis, tinea pedis, onychomycosis, perionychomycosis, pityriasis versicolor, oral thrush, and other candidoses such as vaginal, respiratory tract, biliary, eosophageal, and urinary tract candidoses. Systemic mycoses include systemic and mucocutaneous candidosis, cryptococcosis, aspergillosis, mucormycosis (phycomycosis), paracoccidioidomycosis, North American blastomycosis, histoplasmosis, coccidioidomycosis, and sporotrichosis. Fungal infections include those caused by Cladosporium cucumerinum, Epidermophyton floccosum, Aspergillus fumigatus, and other Aspergillus spp., Rhizopus spp. and Microspermum ypseum.

As will be recognised by those skilled in the art the compounds of the invention can be usefully incorporated in a varied range of compositions. For example, the compounds can be formulated for administration into an animal, including humans, or incorporated in a range of personal care products including body care and oral care such as deodorants, soaps, shampoos, dentifrices, mouthwashes etc. Suitable formulations for use in the methods of the invention can readily be prepared by the skilled addressee using standard procedures.

The present invention also provides for a cleaning composition for cleaning surfaces, for example hard surfaces, woven or unwoven surfaces. Examples of surfaces which may be cleaned and/or cleaning compositions of the invention include toilet bowls, bath tubs, drains, high chairs, countertops (such as those exposed to meats, vegetables), meat processing rooms, butcher shops, airducts, airconditioners, carpets, paper or woven product treatment, diapers and healthy air machines.

The cleaning product may be in the form of a toilet drop-in for prevention and removal of soil and under rim cleaner for toilets.

In another embodiment the compositions will find application as washing solutions, particularly in contact lens cleaning compositions. Thus contact lenses can be cleaned and disinfected by administering a composition comprising an effective amount a compound of the invention.

The compound of the invention may be incorporated into personal care products and skin care products. The compounds of the invention may be used in the preparation of epidermal bandages and lotions. In an alternative embodiment, the compounds of the invention may be incorporated into, for example, aftershaves or lotions.

A pharmaceutical and/or veterinary formulation of the invention comprises suitable “excipients” such that the formulation can be administered to an animal, preferably a human.

Also provided are compositions which can be applied to plants without having a detrimental affect upon the plant.

In another aspect, the composition may comprise a suitable diluent wherein the composition may actually be toxic to, for example, an animal. Such compositions can be useful in industrial settings for disinfecting surfaces etc. Suitable diluents would be well known by those skilled in the art.

In one embodiment, the compounds of the invention are formulated as an emulsion. Emulsions are finely divided or colloidal dispersions comprising two immiscible liquids or “phases”, e.g. oil and water, one of which (the internal or discontinuous phase) is dispersed as droplets within the other (external or continuous phase). Thus, an oil-in-water emulsion consists of oil as the internal phase and water as the external or continuous phase, the water-in-oil emulsion being the opposite.

A wide variety of emulsified systems may be formed comprising a compound of the invention and using microfluidizing technology including standard emulsions and microemulsions.

Generally, emulsions comprise oil and water phases, emulsifiers, emulsion stabilizers, and optionally thickening agents, preservatives, colouring agents, flavouring agents, pH adjusters and buffers, chelating agents, vitamins, anti-foam agents, tonicity adjusters and anti-oxidants. Suitable emulsifiers include (wherein bracketed numerals refer to the preferred hydrophile-lipophile balance (HLB) value): anionic surfactants such as alcohol ether sulfates, alkyl sulfates (30-40), soaps (12-20) and sulfosuccinates; cationic surfactants such as quarternary ammonium compounds; zwitterionic surfactants such as alkyl betaine derivatives; amphoteric surfactants such as fatty amine sulfates, difatty amine sulfates, difatty alkyl triethanolamine derivatives (16-17); and nonionic surfactants such as the polyglycol ether derivatives of aliphatic or cycloaliphatic alcohols, saturated fatty acids and alkylphenols, water-soluble polyethyleneoxy adducts onto polypropylene glycol and alkyl polypropylene glycol, nonylphenol polyoxyethanols, castor oil polyglycol ethers, polypropylene/polyethylene oxide adducts, tributylphenoxy-polyethoxyethoxy-ethanol, lanothin alcohols, polyethylated (POE) alkyl phenols (12-13), POE fatty esters poloxamers (7-19), POE glycol monoethers (13-16), polysorbates (17-19) and sorbitan esters (2-9). This list is not intended to be exhaustive as other emulsifiers are suitable.

In another embodiment, a compound of the invention is formulated in an aqueous composition comprising a water miscible solvent. Examples, of such water miscible solvents include, but are not limited to, ethanol, isopropanol, diethylene glycol monomethyl ether, diethylene glycol butyl ether, diethylene glycol monoethyl ether, diethylene glycol dibutyl ether, polyethylene glycol-300, polyethylene glycol-400, propylene glycol, glycerine, 2-pyrrolidone, N-methyl 2-pyrrolidone, glycerol formal, dimethyl sulfoxide, dibutyl sebecate, polysorbate 80, and mixtures thereof

Dosage forms of pharmaceutical preparations containing a compound of the present invention are appropriately selected according to the administration route and can be prepared by conventional preparation methods. Typically, the compound is formulated for administration by any of the commonly used routes such as oral, nasal, rectal, vaginal, intramuscular, intraveneous administration routes.

For convenience, it is preferred that for human or veterinary uses the compound is formulated for oral administration, wherein the compound or pharmaceutically/veterinary-acceptable salt thereof may be in admixture with commonly known binding materials and excipients. Examples of dosage forms for oral administration include tablets, powders, granules, capsules, solutions, syrups, elixirs, and oily or aqueous suspensions. The compound can be administered to animals orally either directly or by mixing with feedstuff, or in a dissolved form directly given to animals or by mixing with water or feedstuff.

Injectable preparations may contain adjuvants, such as stabilizers, antiseptics, and solubilizers. The injectable solution which may contain these adjuvants may be put into a container and solidified by, for example, lyophilization to prepare a solid preparation which is dissolved on use. The container may contain either a single dose or multiple doses.

Preparations for external application include solutions, suspensions, emulsions, ointments, gels, creams, lotions, and sprays.

Solid preparations may contain, in addition to the active compound, pharmaceutically acceptable additives. For example, the active compound is mixed with additives selected according to necessity from among fillers, extenders, binders, disintegrators, absorption accelerators, wetting agents, and lubricants and formulated into solid preparations.

Liquid preparations include solutions, suspensions, and emulsions. They may contain adjuvants, such as suspending agents, emulsifiers, and so forth.

For veterinary use, the compound can be formulated into powders, fine granules, soluble powders, syrups, solutions, and injections according to the customary methods in the art.

For use as drugs for humans, the dose of the compound can be in the range of from 1 mg to 1 g, and preferably from 100 mg to 300 mg, per day for an adult.

For veterinary use, the dose is generally in the range of from 1 to 200 mg, and preferably from 5 to 100 mg, per kg of body weight per day while varying depending on the purpose of administration (for therapy or for prevention), the kind and the size of the animal, the kind of the pathogenic organisms, and severity of symptom.

The above-mentioned daily doses can be given once a day or in 2 to 4 divided doses. If necessary, a daily dose may exceed the above-specified range.

### EXAMPLE

Methods and Materials

Screening of Crude Extracts

Extracts of the worker and soldier castes of the nasute termite species Nasutitermes exitiosus (Hill) (Isoptera:Termitidae) were screened against Bacillus subtilis (ATCC Strain 6633) at a concentration of 4.0 mg/ml in a bioassay. The assay, which was modified from the one described in WO 01/90035, was designed to detect weak antimicrobial activity. The only difference between the assay used in the current work and that described previously (WO 01/90035) is that in the current case the density of bacterial cells in the Luria-Bertani agar medium was ten times lower than that described in the earlier specification. The modified assay was approximately 2.5 times more sensitive than the original method.

Separation of Antimicrobial Components by Reverse-Phase HPLC

Twelve mL of a 70% methanol extract of N. exitiosus soldiers (containing 86 mg of solute) was fractionated by reverse-phase HPLC using a semi-preparative column at a flow rate of 4 ml/min and a gradient from water containing 0.05% (v/v) trifluroacetic acid to 100% acetonitrile over 20 minutes and then 100% acetonitrile for a further 15 minutes. The UV absorbance of the eluant was monitored at 230 nm. Fractions were collected over 35 minutes. Corresponding fractions were pooled across replicate separation runs and the pooled fractions were evaporated to dryness under nitrogen. Residues of the fractions were redissolved in HPLC grade water (fractions from 1-8 minutes), 70% aqueous methanol (9-15 minutes), or methanol (16-35 minutes). A quantity of each pooled fraction, corresponding to 20 μL of crude extract, was assayed for antimicrobial activity against B. subtilis.

Three fractions showed antimicrobial activity, F1 (22-23 minutes), F2 (23-23.5 minutes), and F3 (23.5-24 minutes), and were analysed by HPLC, mass spectroscopy and NMR spectroscopy.

The HPLC was a System Gold, Model 126, Beckman Instruments, Inc., USA.

The semi-preparative column was a 250×10 mm C18 ODS-AQ, 5 μm, made by YMC Co. Ltd and was sourced from Sapphire Biosciences, Australia

Purity Assessment by Reverse-Phase HPLC

10 μl of a selected fraction or 20 μl of crude extract were injected onto an analytical reverse-phase HPLC column. The adsorbed material was eluted at a flow rate of 0.55 ml/min using the same elution conditions as described above. The eluant was monitored by UV absorbance at 230 nm and by an evaporative light-scattering detector (ELSD) operating at 95° C.

The analytical column was a 250×3 mm C18 ODS-AQ, 5 μm, made by YMC Co. Ltd and was sourced from Sapphire Biosciences, Australia.

Isolation of the Antimicrobial Components of F2 by Reverse-Phase HPLC

Active fraction F2, which was isolated from the extract of the soldiers of N. exitiosus, was further separated by semi-preparative reverse-phase HPLC using isocratic elution with acetonitrile:water (containing 0.05% (v/v) trifluroacetic acid) 55:45 over 50 minutes. Four UV-absorbing peaks, which eluted with retention times of 24-26.5 minutes, 30-32 minutes, 34-36 minutes, and 36.5-39 minutes, were collected. Only one of these fractions, that eluting at 24-26.5 minutes, was biologically active. This was evaporated to dryness for further analysis.

Following this isocratic separation, the biologically active subfraction eluting between 24 and 26.5 minutes was still a mixture of at least two compounds as indicated by ¹H NMR spectroscopy (not shown). This sub-fraction was further separated using the same isocratic system, i.e. acetonitrile:water (0.05% (v/v) trifluroacetic acid) 55:45, but in this case over 30 minutes. Two UV-absorbing peaks designated Q and R, which eluted with retention times of 19.3-21.5 minutes and 23-26 minutes, were collected. Each of these fractions was biologically active and was evaporated to dryness for further analysis.

Isolation of the Antimicrobial Components of F3 by Reverse-Phase HPLC

Active fraction F3 isolated from the extract of the soldiers of N. exitiosus was not homogeneous, and was further separated by reverse-phase HPLC using the semi-preparative column and isocratic elution with acetonitrile: water (0.05% (v/v) trifluroacetic acid) 55:45 over 50 minutes. Four UV-absorbing peaks, which eluted with retention times of 21-23 minutes, 23-24.5 minutes, 25-27 minutes, and 30-32 minutes, were collected. The first, second, and fourth eluting fractions, containing compounds designated as S, T and U respectively, were biologically active and were evaporated to dryness for further analysis.

Results

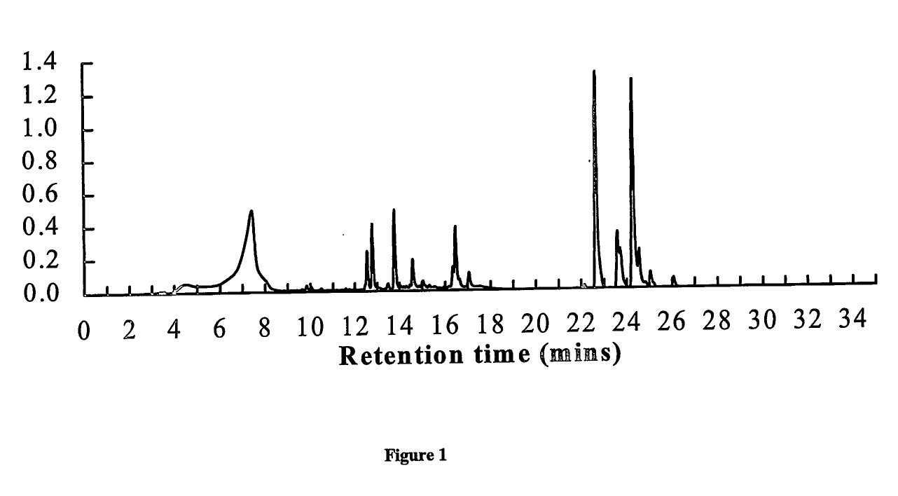

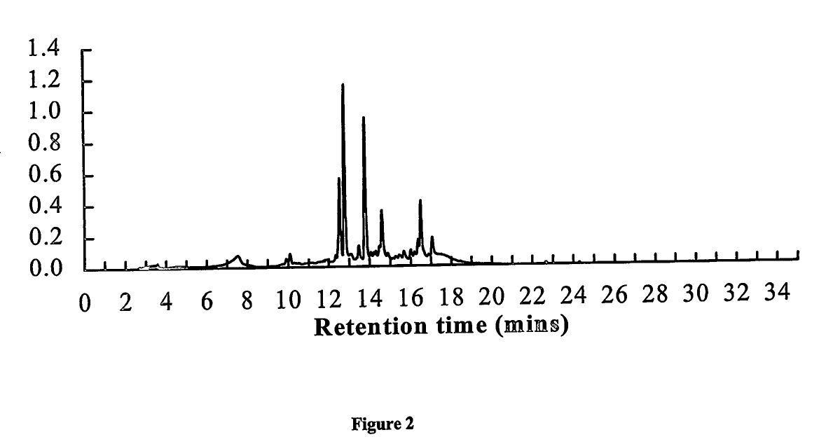

The extract prepared from workers of N. exitiosus was inactive in the modified bioassay, whereas the extract of soldiers of the same species gave a clear zone of 15 mm diameter. The UV and ELSD (not shown) profiles of the two extracts on HPLC were qualitatively very similar between retention times 0-21 minutes, although the heights of the corresponding peaks varied (compare FIGS. 1 and 2). However, major differences between the two extracts were seen between retention times 22 and 27 minutes. Several prominent peaks in this region were detected by UV absorbance at 230 nm in the extract of soldiers (FIG. 1), but no corresponding peaks appeared in the extract of workers of N. exitiosus (FIG. 2). Trinervitane derivatives would be expected to migrate in this region.

Following separation of 12 mL of an extract of N. exitiosus soldiers as described in the Materials and Methods, three fractions F1, F2 and F3 showed antimicrobial activity. F1 gave a faint clear zone of 4 mm diameter in the antimicrobial assay, F2 gave a faint clear zone of 6 mm diameter and F3 contained the highest level of activity giving a very clear zone of 9 mm diameter. The three fractions were analysed by HPLC, mass spectroscopy and NMR spectroscopy.

Structure of P, an Antimicrobial Trinervitatetraene

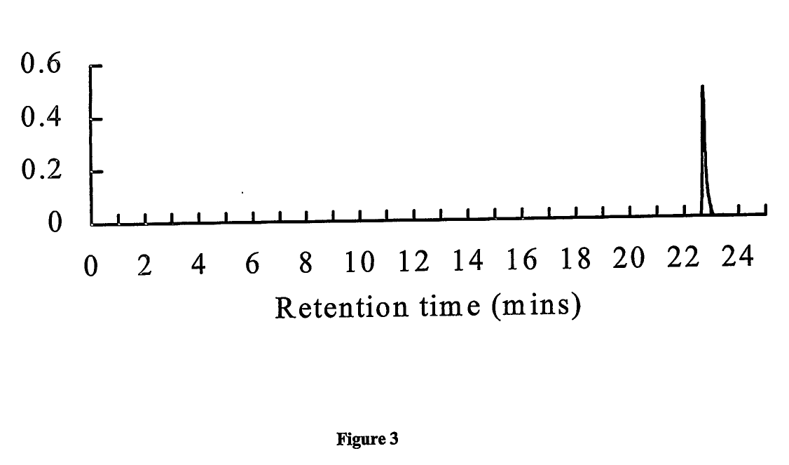

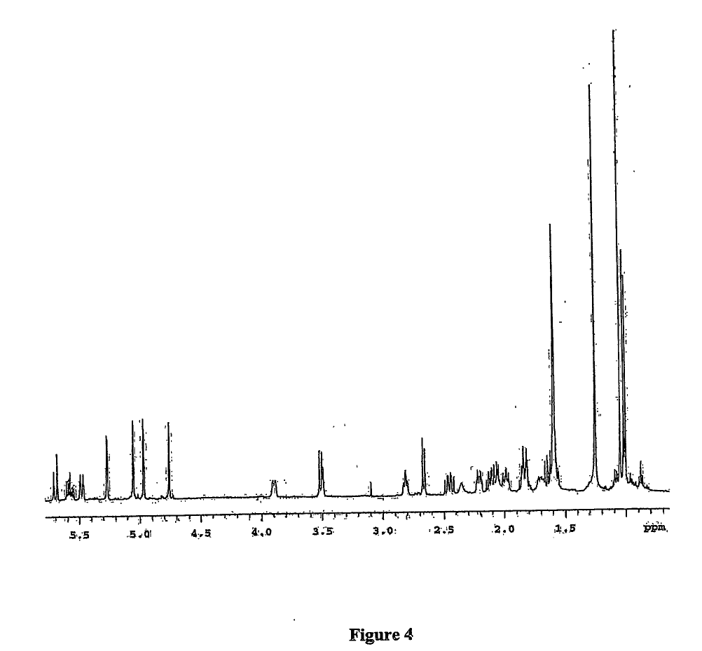

Antimicrobial fraction F1 was found to contain a pure compound, designated P, as shown by HPLC analysis (FIG. 3) and ¹H NMR spectroscopy (FIG. 4).

Approximately 3 mg of compound P was available. ESMS of the compound showed ions at m/z 323 and 623 respectively, corresponding to sodiated monomeric and dimeric forms of a species of molecular weight 300 amu. EIMS showed a molecular ion at m/z 300, and a daughter ion, due to the loss of one molecule of water, at m/z 282. HREIMS established the molecular formula as C₂₀H₂₈O₂.

APT spectroscopy defined the type of carbon and the extent of its substitution for all 20 carbon atoms. Proton resonances were then assigned to the respective carbon atoms by HMQC spectroscopy, as listed in Table 1.

TABLE 1
¹³C and ¹H NMR spectral data for compound P (in CDCl₃).

| Position | δ C | δ H | C-type |
|---|---|---|---|
| 1 | 137.8 | — | C═ |
| 2 | 73.0 | 3.90(ddd, 10.5, 2.0) | CH(OH) |
| 3 | 79.0 | 3.51(d, 10.5) | CH(OH) |
| 4 | 45.8 | — | C |
| 5 | 37.7 | 2.12(m), 1.58^{a}(m) | CH₂ |
| 6 | 29.4 | 1.97(m), 1.83^{a}(m) | CH₂ |
| 7 | 52.0 | 2.82(t, 7.5) | CH |
| 8 | 149.7 | — | C═ |
| 9 | 132.4 | 5.70(d, 15.0) | CH═ |
| 10 | 130.1 | 5.58(ddd, 15.0, 10.0, 5.5) | CH═ |
| 11 | 42.3 | 2.20^{a}(m), 1.61^{a}(m) | CH₂ |
| 12 | 33.8 | 1.70^{b}(m) | CH |
| 13 | 36.1 | 2.46(m), 1.84^{a}(m) | CH₂ |
| 14 | 124.6 | 5.48(dt, 11.5, 2.5) | CH═ |
| 15 | 141.8 | — | C═ |
| 16 | 60.7 | 2.66(d, 8.0) | CH |
| 17 | 120.3 | 5.27(d, 2.5), 4.97(d, 2.5) | CH₂═ |
| 18 | 21.3 | 1.05(s) | CH₃ |
| 19 | 108.1 | 5.05(s), 4.76(s) | CH₂═ |
| 20 | 23.4 | 1.01(d, 7.0) | CH₃ |

^{a}Data from COSY spectrum.
^{b}Data from HMQC spectrum.

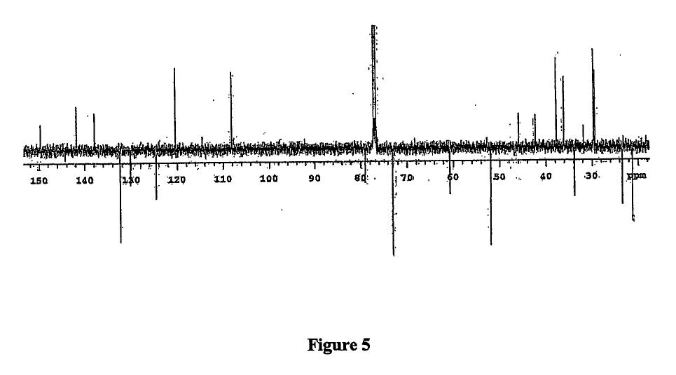

The APT spectrum (FIG. 5) defined eight olefinic carbon atoms at δ 149.7, 141.8, 137.8, 132.4, 130.1, 124.6, 120.3 and 108.1. Since there were no other unsaturated carbon atoms, compound P must be tricyclic. The two pairs of olefinic protons, δ 5.27 and 4.97, and 5.05 and 4.76, found in ¹H NMR spectrum (FIG. 4) were shown by HMQC spectroscopy (Table 1) to be attached to the δ 120.3 and 108.1 carbons respectively as two methylene groups ═CH₂. These data and the molecular formula suggested that the compound had the 8(19),15(17)-trinervitadiene carbon skeleton V with two additional olefinic bonds and two hydroxyl groups. No compounds with the molecular formula C₂₀H₂₈O₂ and carbon skeleton V have been reported previously.

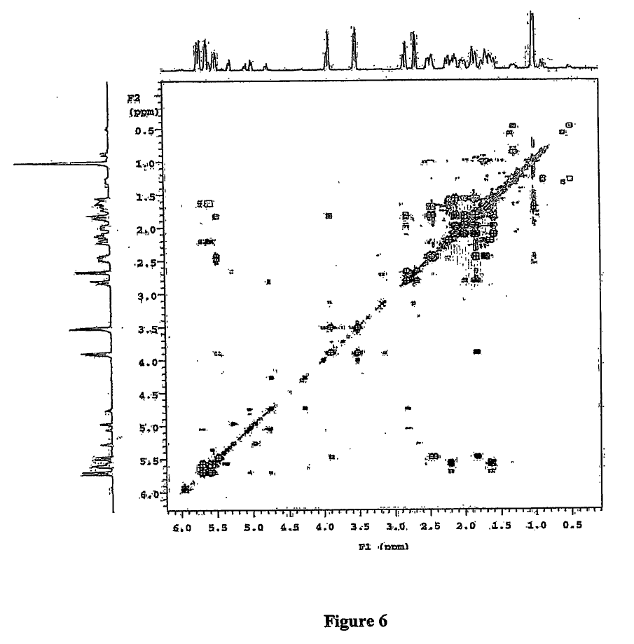

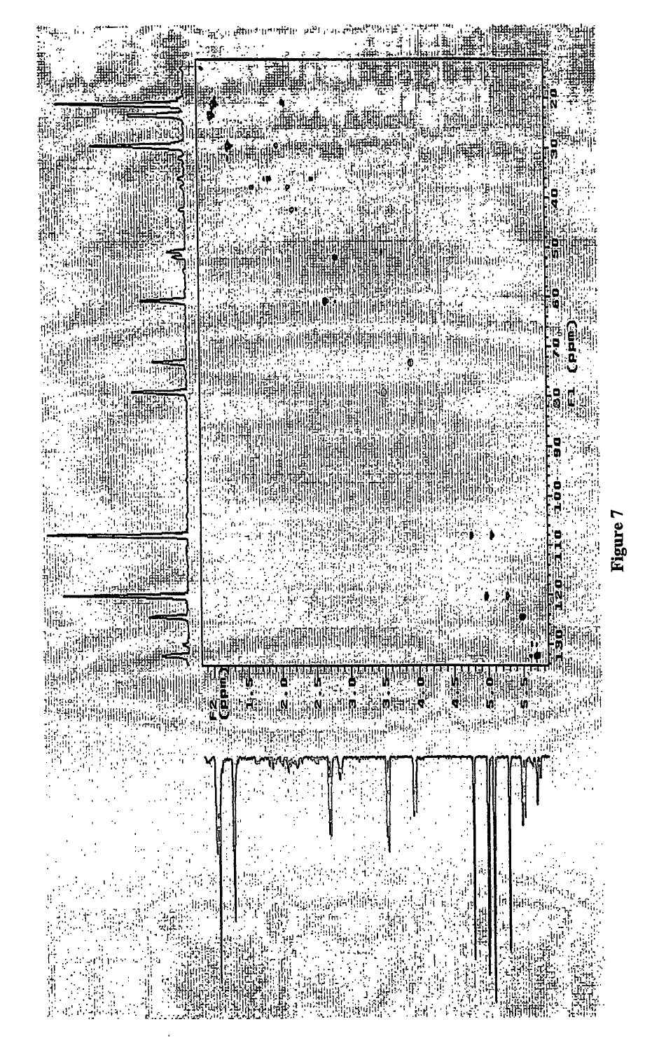

Compound P showed three further olefinic protons, δ 5.70, 5.58, and 5.48, in the ¹H NMR spectrum. The connections between these protons and the olefinic carbons at δ 132.4, 130.1, and 124.6 seen in the APT spectrum were established by HMQC spectroscopy (Table 1) as three olefinic methine carbons (═CH—). The two protons, δ 5.70 and 5.58, were mutually coupled with J=15.0, implying a E-disubstituted olefin [3], which was supported by COSY spectroscopy (FIG. 6). The two hydroxyl bearing methine carbons —CHOH— (δ 79.0 and 73.0) seen in the APT spectrum were connected to the two mid-field resonances at δ 3.51 and 3.90 in ¹H NMR spectrum by HMQC spectroscopy (FIG. 7). The signals of the two protons were mutually coupled with J=10.5, confirmed by COSY spectroscopy, indicating a vicinal diol segment —CH(OH)CH(OH)—.

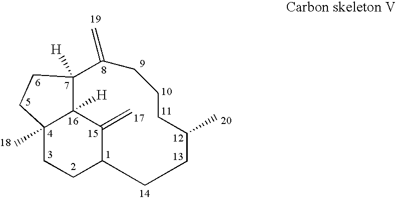

COSY spectroscopy established that the methine proton δ 2.66 was coupled to an adjacent methine proton at δ 2.82 with J=8.0, which then formed a linear 6-spin system —CHCHCH₂CH₂— with the protons of two consecutive methylene groups at δ 1.97 and 1.83, and 2.12 and 1.58. The olefinic proton δ 5.58 was coupled to adjacent methylene protons at δ 2.20 and 1.61, which in turn completed an 11-spin system —CH═CHCH₂CH(CH₃)CH₂CH═ with consecutive protons at δ 1.70 (1.01), 2.46 and 1.84, and 5.48. These 6- and 11-spin systems clearly constitute the C-16,7,6,5 and C-9 to C-14 segments of the 5-membered ring and the medium ring, respectively, in carbon skeleton V. The segment of the medium ring was oriented by long range coupling effects. The olefinic proton δ 5.70 was positioned at C-9, in accordance with allylic coupling seen in the COSY spectrum between δ 5.70 and the methylene protons at δ 5.05 and 4.76, which in turn were allylically coupled with the methine proton δ 2.82 at C-7. The olefinic proton δ 5.48 was positioned at C-14, in accordance with allylic coupling seen in the COSY spectrum between δ 5.48 and 3.90, a proton of the vicinal diol (—CH(OH)CH(OH)—) which was clearly the C-2,3 segment of the 6-membered ring.

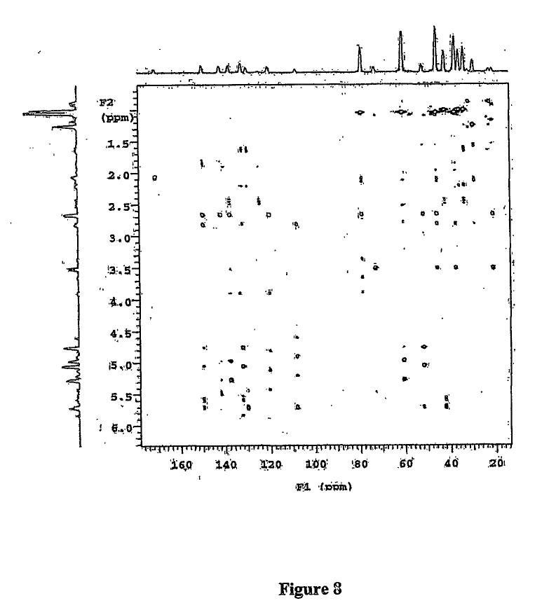

Conclusive proof of carbon skeleton V, and of the connection between all the above segments, was obtained from numerous long range H—C correlations observed in HMBC spectroscopy (FIG. 8). In particular, the position of the substituents in the five and six membered rings follow from 3-bond couplings between CH(OH)-3 and C-1, C-5 and C-18, CH₃-18 and C-3, C-5 and C-16, CH-7 and C-4 and C-5, CH-16 and C-3 and C-17, CH-9 and C-11, CH₂-17 and C-16, and CH₃-20 and C-13. The point of attachment of the medium ring to the 5-membered ring follows from reciprocal 3-bond H,C couplings between CH-7, CH-9 and CH₂-19 and coupling between CH-16 and C-8. The point of attachment of the medium ring to the 6-membered ring system follows from 3-bond H,C couplings between CH-14 and C-15, and CH-16 and C-1. Compound P was thus identified as 9E-1(14),8(19),9(10),15(17)-trinervitatetraene-2,3-diol, without regard to stereochemistry except the alkene at 9(10). No trinervitatetraene has been reported previously.

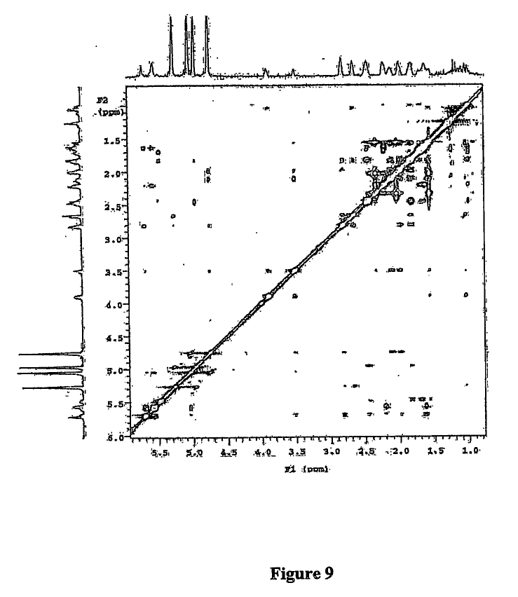

The stereochemistry of P was studied by NOESY (FIG. 9) and GOESY spectroscopy. Both types of NOE spectroscopy showed proximity between CH-7, CH-16 and CH₃-18, and they are assumed to be a-oriented in accordance with the normal carbon skeleton V. Mutual Overhauser effects were also observed between CH-2 and CH₃-18, indicating CH-2 as α-oriented, and thus the hydroxyl group at C-2 as β-oriented. The configuration of the hydroxyl group at C-3 was deduced from the value of its vicinal coupling constant and by analogy with data in the literature [4]. The value J_{2,3}=10.5 Hz indicated a trans-diaxial arrangement of protons at C-2 and C-3 (such trans-diols have J_{2,3}=8.0-10.0 Hz while cis-diols have J_{2,3}=4.5-5.0 Hz [4-6]). Therefore the hydroxyl group at C-3 is α-oriented. No correlation was seen for the CH₃ at C-12, but it was assumed to be α-oriented, in accordance with the methyl group at C-12 of all reported trinervitanes [4,7].

All the reported trinervitanes have been assumed to have the absolute configuration depicted in carbon skeleton V [8,9]. The relative configuration of P defined by Overhauser effects was therefore also assumed to represent the absolute configuration, i.e., 9E-1(14),8(19),9(10),15(17)-trinervitatetraene-2β,3α-diol as in structure P.

Structure of Q, a Novel Antimicrobial Compound

Fraction F2 was a mixture, which contained at least two major and two minor components as indicated by four peaks on HPLC and a mixture evident in ¹H NMR spectroscopy. The fraction was further separated into two major biologically active components (as described in Materials and Methods), designated Q and R, which gave clear zones with diameters of 4 mm and 5 mm respectively in the modified antimicrobial assay (Materials and Methods). These compounds were pure as judged by ¹H NMR spectroscopy.

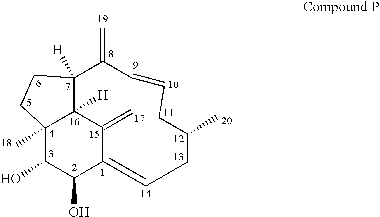

An estimated 0.5 mg of compound Q was obtained. EIMS showed a molecular ion at m/z 302, and a daughter ion due to the loss of one molecule of water at m/z 284. HREIMS established the molecular formula as C₂₀H₃₀O₂.

APT spectroscopy defined four olefinic carbon atoms at δ 138.0, 123.8, 118.4 and 106.7 (Table 2). Five olefinic protons, δ 5.55, 5.22, 4.98, 4.91 and 4.79, were apparent in the ¹H NMR spectrum (Table 2). The two pairs of olefinic protons, δ 5.22. and 4.98, and 4.91 and 4.79 were shown by HMQC spectroscopy to be attached to the δ 118.4 and 106.7 carbons respectively as two methylene groups ═CH₂ (Table 2). The fifth olefinic proton δ 5.55 was not coupled with these methylene protons, but was shown by the HMQC spectroscopy to be attached to the δ 123.8 carbon as an olefinic methine group (═CH—) (Table 2), implying the presence of three alkenes in this compound. The compound Q was thus tricyclic. These data in conjunction with the molecular formula suggested that the compound had the 8(19),15(17)-trinervitadiene carbon skeleton V with an additional olefinic bond and two hydroxyl groups. No compounds with both the molecular formula C₂₀H₃₀O₂ and carbon skeleton V have been reported previously.

The ¹H NMR spectrum showed two mid-field resonances at δ 3.93 and 3.76 (Table 2). The two hydroxyl bearing methine carbons —CH(OH)— (δ 73.9 and 77.0) seen in the APT spectrum were connected to the resonances at δ 3.93 and 3.76 by HMQC spectroscopy (Table 2). The signals of the two protons were mutually coupled with J=10.0, which was supported by COSY spectroscopy, indicating a vicinal diol segment —CH(OH)CH(OH)—. COSY spectroscopy (Table 2) established that the methine proton δ 5.55 was coupled to adjacent methylene protons at δ 2.63 and 1.99, forming a segment >C═CH—CH₂—. Homoallylic coupling was seen in the COSY spectrum between the protons at δ 1.99 and 3.93, implying a segment —CH(OH)CH(OH)—C═CH—CH₂—, which clearly constitutes the C-3,2,1,14,13 segment of carbon skeleton V. Two methine carbons (3 51.1 and 61.3) seen in the APT spectrum were assigned to C-7 and C-16 by comparison with those of P (δ 52.0 and 60.7), since the corresponding carbon atoms of the two compounds are in similar chemical environments. The COSY spectrum was complicated by overlapping proton signals at approximately δ 2.65, 2.20, and in the δ 1.2-1.7 region, and could not provide further useful correlations.

TABLE 2
¹³C and ¹H NMR spectral data for compound Q (in CDCl₃).

| Position | δ C | δ H |
|---|---|---|
| 1 | 138.0^{c} | — |
| 2 | 73.9 | 3.93(dm, 10.0) |
| 3 | 77.0^{b} | 3.76(d, 10.0) |
| 4 | u | — |
| 5 | u | u |
| 6 | u | u |
| 7 | 51.1 | 2.63 or 2.67(m) |
| 8 | u | — |
| 9 | u | u |
| 10 | u | u |
| 11 | u | u |
| 12 | u | 1.82(m) |
| 13 | u | 2.63(m), 1.99(m) |
| 14 | 123.8 | 5.55(dt, 12.5, 2.5) |
| 15 | u | — |
| 16 | 61.3 | 2.63 or 2.67(m) |
| 17 | 118.4 | 5.22(d, 3.0), 4.98(d, |
| | | 3.0) |
| 18 | 20.6 | 1.04(s) |
| 19 | 106.7 | 4.91(s), 4.79(s) |
| 20 | 21.3^{b} | 0.99(d, 7.0) |

^{b}Data from HMQC spectrum.
^{c}Data from HMBC spectrum.
u: Data unresolved.

Conclusive proof of the presence of skeleton V, and of the connections between all the above segments, was obtained from a number of long range H,C correlations recorded by HMBC spectroscopy. In particular, the position of the substituents in the 6,5-membered rings follows from reciprocal 3-bond H,C couplings between CH(OH)-3 and CH₃-18, coupling between CH₃-18 and C-16, and CH₂-17 and C-16. The point of attachment of the medium ring to the 5-membered ring system follows from 3-bond H,C coupling between CH₂-19 and C-7. The point of attachment of the medium ring to the 6-membered ring system follows from coupling between CH₂-17 and C-1, confirming the segment, —CH(OH)CH(OH)—C═CH—CH₂—. Compound Q was thus identified as 1(14),8(19),15(17)-trinervitatriene-2,3-diol, without regard to stereochemistry.

The stereochemistry of Q was investigated by NOESY and GOESY spectroscopy. Both types of NOE spectroscopy showed proximity between CH-2 and CH₃-18, and CH-7 and CH-16. As CH-7, CH-16, and CH₃-18 are assumed to be α-oriented in accordance with the carbon skeleton V, CH-2 is thus α-oriented and the hydroxyl group at C-2 is β-oriented. The configuration of the hydroxyl group at C-3 was deduced from the value of its vicinal coupling constant and from analogy with literature data [4]. The value J_{2,3}=10.0 Hz indicated a trans-diaxial arrangement of protons at C-2 and C-3 (such trans-diols have J_{7,3}=8.0-10.0 Hz while cis-diols have J_{2,3}=45-5.0 Hz [4-6]). Hence, the hydroxyl group at C-3 is α-oriented. This was confirmed by comparing the chemical shifts of the protons at C-2 and C-3 with those of compound P which has 2β,3α diol functionality. The δ 3.93 proton at C-2 was similar to that of P (δ 3.90) while the δ 3.76 proton at C-3 was shifted 0.25 ppm to lower field than that of P, possibly due to the lack of shielding by the 9(10)-alkene in P. In the compound P, the proximity between CH-3 and the olefinic proton CH-9 was shown by NOESY spectroscopy. No correlation was seen for the CH₃ at C-12 in compound Q, but, it was assumed to be a-oriented, in accordance with all previously reported trinervitanes [4,7].

As argued previously, all the reported trinervitanes have been assumed to have the absolute configuration depicted in carbon skeleton V [8,9]. The relative configuration of this compound Q defined by Overhauser effects was therefore also assumed to represent the absolute configuration, i.e., 1(14),8(19),15(17)-trinervitatriene-2β,3α-diol as in structure Q. 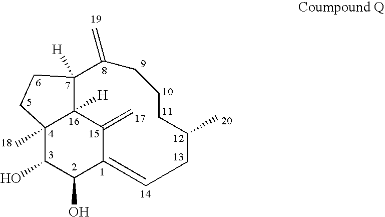
Structure of R, a Novel Animation Compound

An estimated 0.3 mg of the pure compound R was obtained by refractionation of F2 (Materials and Methods). EIMS showed a molecular ion at m/z 302 and a daughter ion due to the loss of one molecule of water at m/z 284. HREIMS established the molecular formula as C₂₀H₃₀O₂, isomeric with Q.

The NMR data for compound R was limited by the small quantity of sample available. The ¹H NMR spectrum showed only three olefinic protons, δ 5.51, 5.16, and 4.97, and a broad singlet at δ 1.47 of an olefinic methyl group (Table 3). The two olefinic protons, δ 5.16 and 4.97, were shown by HMQC spectroscopy to be attached to a carbon atom at δ 117.0 as a methylene group ═CH₂ (Table 3). The olefinic proton δ 5.51 was not coupled with these methylene protons as shown by the COSY spectrum. The olefinic methyl signal showed only homoallylic coupling with the proton at δ 2.89 but no correlations to the three olefinic protons, indicating that three alkenes were present in this compound, which was thus tricyclic.

TABLE 3
¹³C and ¹H NMR spectral data for compound R (in CDCl₃).

| Position | δ C | δ H |
|---|---|---|
| 1 | u | — |
| 2 | 74.4^{b} | 3.97(dm, 10.0) |
| 3 | 74.7^{b} | 3.27(d, 10.0) |
| 4 | 49.0^{c} | — |
| 5 | 34.0^{c} | u |
| 6 | u | u |
| 7 | u | — |
| 8 | u | — |
| 9 | u | u |
| 10 | u | u |
| 11 | 27.0^{c} or 33.0^{c} | u |
| 12 | u | u |
| 13 | 27.0^{c} or 33.0^{c} | 2.61(dt, 12.0), 1.80(bd, |
| | | 15.5) |
| 14 | u | 5.51(dm, 12.0) |
| 15 | u | — |
| 16 | 60.0^{b} | 2.89(m) |
| 17 | 117.0^{b} | 5.16(d, 2.5), 4.97(d, 2.5) |
| 18 | 17.5^{b} | 1.02(s) |
| 19 | 17.7^{b} | 1.47(bs) |
| 20 | 20.4^{b} | 0.96(d, 6.5) |

^{b}Data from HMQC spectrum.
^{c}Data from HMBC spectrum.
u Data unresolved.

Although two olefinic methylenes (CH₂═) were present in compounds P and Q, only one such methylene was seen in compound R, possibly implying an alkene shifted from >C═CH₂ at 8(19) in carbon skeleton V into >C═C(CH₃)— at 7(8) or —(CH₃)C═CH— at 8(9), or from >C═CH₂ at 15(17) into >C═C(CH₃)— at 1(15) or 15(16). The chemical shift of the olefinic methylene carbon atom of R (δ 117.0) was similar to those at C-17 of P (δ 120.3) and Q (δ 118.4), but not to those at C-19 of P (δ 108.1) or Q (δ 106.7), confirming the presence of a 15(17)-exomethylene function. If the alkene shifted from >C═CH₂ at 8(19) into —(CH₃)C═CH— at 8(9), allylic coupling would be expected. Furthermore, the resulting olefinic methine proton would be expected to be coupled with two adjacent allylic protons as a doublet of doublets with coupling constant values similar to 6.0 and 11.0 Hz as shown in compound 9 of WO 01/90035. No such proton was apparent in the ¹H NMR spectrum of R. The δ 5.51 proton was instead coupled with two adjacent protons as a doublet of multiplets with J=12.0, resembling the C14,13 segment ═CHCH₂— of compounds P and Q and indicating the alkene was possibly shifted from 8(19) of skeleton V into 7(8) as in carbon skeleton W. These data and the molecular formula suggest that compound R has the trinervitadiene carbon skeleton W with an additional olefinic bond and two hydroxyl groups [4,8]. There have been no reports of natural products with the molecular formula C₂₀H₃₀O₂ and skeleton W, but a trinervitane of this type has been synthesised previously [10]. The ¹H NMR characteristics of the latter compound 7(8),11(12),15(17)-trinervitatriene-2α,3α-diol differ significantly from those of R, which is thus a novel compound.

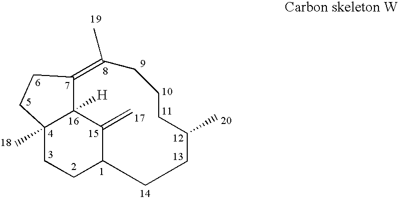

The ¹H NMR spectrum showed two mid-field resonances at δ 3.97 and 3.27 (Table 3). Two hydroxyl bearing methine carbons —CH(OH)— (δ 74.4 and 74.7) were connected to these resonances by HMQC spectroscopy (Table 3). The signals of the two protons were mutually coupled with J=10.0, supported by COSY spectroscopy, indicating a vicinal diol segment —CH(OH)CH(OH)—. COSY spectroscopy (Table 3) established that the methine proton δ 5.51 was coupled to adjacent methylene protons δ 2.61 and 1.80, forming a segment >C═CH—CH₂—. Homoallylic coupling was seen in the COSY spectrum between the protons δ 1.80 and 3.97, implying a segment —CH(OH)CH(OH)—C═CH—CH₂—, which clearly constitutes the C-3,2,1,14,13 segment of carbon skeleton W. The homoallylic coupling seen in the COSY spectrum between δ 2.89 and the olefinic methyl protons δ 1.47 implied a segment CH—C═C—CH₃, which constitutes the C-16,7,8,19 segment of the skeleton W. The proton δ 2.89 was thus assigned to C-16. The COSY spectrum was complicated by overlapping signals of protons at approximately δ 2.65, 2.20, and in the δ 1.2-2.2 region, and could not provide further correlations.

Conclusive proof of the presence of carbon skeleton W, and of the connections in the 6,5-membered rings, was obtained from a number of long range H,C correlations observed in HMBC spectroscopy, particularly the 3- and 4bond couplings between CH3-18 and C-3, C-16, C-5, and C-2. The points of attachment of the medium ring to the 5,6-membered rings follow from the two segments, —CH(OH)CH(OH)—C═CH—CH₂— and CH—C═C—CH₃ determined from homoallylic couplings. The compound R was thus identified as 1(14),7(8),15(17)-trinervitatriene-2,3-diol, without regard to stereochemistry.

The stereochemistry of this compound R was investigated by NOESY and GOESY spectroscopy. Both types of NOE spectroscopy showed proximity between CH₃-18 and CH-16, and they are assumed to be α-oriented, in accordance with carbon skeleton W. The value J_{2,3}=10.0 Hz indicated a trans-diaxial arrangement of protons at C-2 and C-3 (such trans-diols have J_{2,3}=8.0-10.0 Hz while cis-diols have J_{2,3}=4.5-5.0 Hz [4-6]). Hence, the hydroxyl groups at C-2 and C-3 had the configuration βα or αβ. The J_{2,3} value was identical to that of compound Q and similar to that of P (J_{2,3}=10.5 Hz), implying similar 2β,3α diol configurations. This was confirmed by comparing the chemical shifts of the protons at C-2 and C-3 with those of P and Q. The value δ 3.97 for CH-2 was similar to those of trinervitanes P (3.90) and Q (3.93), while CH-3 at δ 3.27 was shifted to higher field than in P (3.51) and Q (3.76), reflecting the altered alkene arrangements in the medium ring. In both P and Q, the proximity between CH-3 and the olefinic protons CH-19 indicated by molecular models was confirmed by NOESY data. The methyl group at C-12 was assumed to be a-oriented, in accordance with the C-12 configuration in all reported trinervitanes [4].

According to the arguments rehearsed previously the absolute configuration of R, 1(14),7(8),15(17)-trinervitatriene-2β,3α-diol, was assumed to be as depicted.

Other Antimicrobial Trinervitane Derivatives Identified in the Extract of Soldiers of N Exitiosus.

Fraction F3 contained at least three major components and one minor component as indicated by four peaks on HPLC and was evidently a mixture from ¹H NMR spectroscopy. The fraction was further separated into three major antimicrobial components (Materials and Methods). Each of these components was pure as judged by its ¹H NMR spectrum. The compounds were identified as 1(15),8(9)-trinervitadiene-2β,3α-diol (S), 1(15),8(9)-trinervitadiene-3α-ol-2-one (C), and 1(15),8(19)-trinervitadiene-2β,3α,9α-triol 2,3,9-triacetate (U).

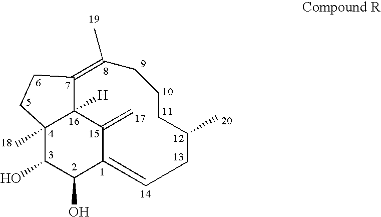

Compound S was identified as 1(15),8(9)-trinervitadiene-2β,3α-diol because it showed an identical retention time on reverse phase HPLC and identical ¹H chemical shifts to compound 9 of WO 01/90035. This compound was shown to have antimicrobial activity in the earlier patent specification.

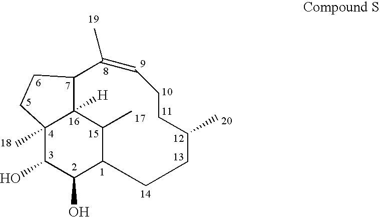

An estimated 0.5 mg of the pure compound T was obtained. EIMS showed a molecular ion at m/z 302, daughter ions due to the loss of a methyl group at m/z 287 and further loss of one molecule of water at m/z 269. HREIMS established the molecular formula as C₂₀H₃₀O₂.

The ¹H NMR spectrum of compound T (Table 4) showed inter alia methyl resonances as a singlet at δ 1.02 and two doublets at 0.67 and 1.42. The olefinic methylene protons present in a number of trinervitanes such as the present compounds P, Q and R and compounds 6, 7 and 8 of WO 01/90035 were not seen in compound T. However, a doublet of doublets with J=10.0 and 5.0 was visible downfield at δ 4.80, similar to the doublet of doublets δ 5.29 with J=11.0 and 6.0 of the olefinic proton at C-9 of compound S (See Table 4 of WO 01/90035), implying an 8(9)-alkene as in the carbon skeleton X. These data and the molecular formula suggested that the compound T had the 1(15),8(9)-trinervitadiene skeleton X with additional hydroxyl and carbonyl groups.

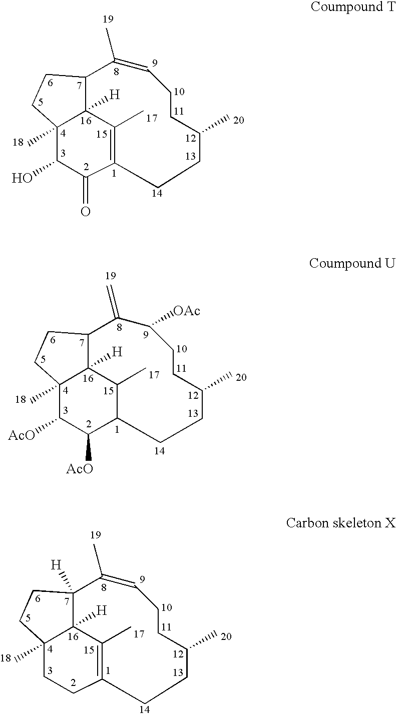

TABLE 4
¹H NMR spectral data for compound T (in benzene d₆).

| | | 1(15),8(9)- |
|---|---|---|
| | | Trinervitadiene-3α-ol- |
| Position | δ H | 2-one δ H* |
| 3 | 4.29(s) | 4.28(s) |
| 9 | 4.80(bdd, 10.0, 5.0) | 4.80(bdd, 10.6, 6.0) |
| 16 | 3.01(bd, 11.5) | 3.01(bd, 12.0) |
| 17 | 1.42(d, 5.5) | 1.42(bs) |
| 18 | 1.02(s) | 1.02(s) |
| 20 | 0.67(d, 7.0) | 0.67(d, 6.6) |

*See reference Valterova et al. [8].

There are four known trinervitanes with the molecular formula C₂₀H₃₀O₂, including the two novel diols Q and R described here and two other compounds, 1(15),8(19)-trinervitadiene-3α-ol-2-one and 1(15),8(9)-trinervitadiene-3α-ol-2-one. The latter compounds have been isolated from a number of termite species of the genera Nasutitermes, Longipeditermes, Lacessititermes, Hospitalitermes, Trinervitermes, and Grallatotermes in the subfamily Nasutitermitinae [4,8,11-19]. The key proton resonances of compound T were identical to those published for 1(15),8(9)-trinervitadiene-3α-ol-2-one (Table 4). T is an antimicrobial compound, confirming the inference to that effect in WO 01/90035.

An estimated 0.5 mg of the pure compound U was obtained. ESMS of the compound showed ions at m/z 469 and 915 respectively, corresponding to sodiated mono- and dimeric forms of a species of molecular weight 446 amu. EIMS showed no molecular ion at this mass but rather daughter ions due to the loss of one, two, and three molecules of acetic acid at m/z 386, 326, and 266. HREIMS established the composition of the first of these daughter ions as C₂₄H₃₄O₄, corresponding to a parent molecular formula C₂₆H₃₈O₆. The ¹H NMR spectrum of compound U (Table 5) showed methyl resonances as four singlets at δ 2.04, 2.08, 2.13 and 1.08, a doublet at 1.74, and a doublet with J=6.5 at 0.89. The first three of these signals correspond to acetate methyl resonances. The spectrum showed five low field protons, at δ 5.58, 5.54, 5.52, 5.31 and 5.24. The signals at δ 5.31 and 5.24 were singlets, and resembled the protons of a methylene group ═CH₂, which typically shows signals in this region. The three remaining low field protons could be acetoxymethine signals —CHOAc—. These data and the molecular formula suggested that the compound U had the 1(15),8(19)-trinervitadiene carbon skeleton Y with three additional acetate groups.

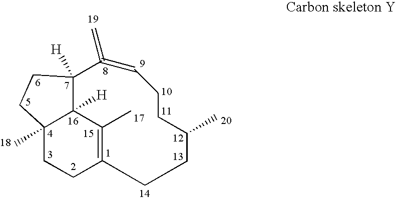

TABLE 5
¹H NMR spectral data for compound U (in CDCl₃).

| | | 1(15),8(19)-Trinervitadiene- |
|---|---|---|
| | | 2β,3α,9α-triol 2,3,9 |
| | | triacetate |
| Position | δ H | δ H* |
| 2 | 5.58(bs) | 5.58(bs) |
| 3 | 5.54(bs) | 5.58(bs) |
| 7 | 3.21(dt, 12, 12, 8) | 3.23(dt, 11, 11, 8) |
| 9 | 5.52(bs) | 5.51(bt, 6, 6) |
| 16 | 2.42(bd, 12) | 2.41(bd, 12) |
| 17 | 1.74(d, 1.5) | 1.75(bs) |
| 18 | 1.08(s) | 1.08(s) |
| 19 | 5.24(s) | 5.23 |
| | 5.31(s) | 5.29 |
| 20 | 0.89(d, 6.5) | 0.89(d, 6.5) |
| OAc | 2.04(s), 2.08(s), | 2.03(s), 2.03(s), 2.12(s) |
| | 2.13(s) | |

*See reference Valterova et al. [8].

Three triacetates of trinervitadiene triols have been reported previously with the molecular formula C₂₆H₃₈O₆. 1(15),8(19)-Trinervitadiene-2α,3α,9α-triol 2,3,9-triacetate was disclosed as Formula 10 of WO 01/90035. 1(15),8(19)-Trinervitadiene-2β, 3α, 13α-triol 2,3,13-triacetate and 1(15),8(19)-trinervitadiene-2β,3α,14α-triol 2,3,14-triacetate have been isolated from a number of termite species of the genera Nasutitermes, Longipeditermes, Lacessititermes, and Hospitalitermes in the subfamily Nasutitermitinae [5,12,14-16]. The key proton resonances of compound U identified it as 1(15),8(19)-trinervitadiene-2β,3α,9α-triol 2,3,9-triacetate. U was isolated on the basis of its antimicrobial activity, which was predicted in WO 01/90035.

Discussion

Tricyclic diterpenes were confined to the extract that was made from soldiers of N. exitiousus, and were not present in the corresponding extract made from workers of the same species. This result confirms a previous observation that only termite soldiers produced trinervitanes [7].

The work described herein details the isolation and structural determination of three novel trinervitanes (P, Q and R) and the identification of another three known trinervitanes (S, T, and U) from the Australian termite N. exitiosus. The three novel trinervitanes, although possessing the relatively common 2β,3α diol oxygenation pattern, had structural features that have not previously been observed amongst the range of compounds with the trinervitane carbon skeletons. All three novel trinervitanes possessed antimicrobial activity. The previously observed antimicrobial activity of compound S was confirmed, as was the antimicrobial activity previously predicted for the compounds T and U.

Table 6 summarises the distribution of double bond variants of trinervitanes within nasute taxonomy. Species from the genus Nasutitermes have previously provided a number of trinervitadienes with the alkene positions commonly at 1(15),8(9) or 1(15),8(19) [7], and occasionally at 11(12),15(17) [13,20]. Trinervitanes with more than two double bonds have not been reported previously in nature. Accordingly antibiotic activity has not previously been observed or imputed to trinervitane derivatives with more than two olefinic bonds. Only one trinervitane with a greater number of double bonds, 7(8),11(12),15(17)-trinervitatriene-2α,3α-diol, has been synthesized [10]. However, compound P has four double bonds while Q and R have three double bonds. The presence of double bonds at the 1(14) and 9(10) positions in P, the 1(14) position of Q, and the 1(14) and 7(8) positions of R are novel among natural trinervitanes, although a trinervitadiene with an alkene in the 7(8) position has been synthesized previously [10,21].

The antimicrobial potency of the compounds P, Q, R, T and U has not been determined quantitatively, due to the relatively small amounts of the compounds. available. However, the screening assays used indicated that P, Q, R, T and U have qualitatively similar levels of antimicrobial activity to those of trinervitadienes previously isolated from N. triodiae

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

TABLE 6
Distribution of number and position of trinervitane double bonds among termite taxa of subfamily Nasutitermitinae.

| Pattern | Double bonds | Position of double bonds | Occurrence and references |
|---|---|---|---|
| 1 | none | N/A | N/A |
| 2 | trinervitenes | 1(15) or 15(17) | Found in: |
| | | | Nasutitermes [13, 24] Hospitalitermes [25, 26] |
| | | | but less common than 1(15), 8(9) and 1(15), 8(19) in pattern 3 |
| 3 | trinervitadienes | 1(15), 8(9) or 1(15), 8(19) | Commonly found, exemplified by: |
| | | | Nasutitermes [4, 5, 7, 13, 16], Trinervitermes [4], Lacessititermes [15], |
| | | | Hospitalitermes [12], Longipeditermes [14] |
| | | 15(17), 11(12) | Found in: |
| | | | Nasutitermes [13, 27] |
| | | | but less common than 1(15), 8(9) and 1(15), 8(19) |
| 4 | trinervitatrienes | 1(14), 8(19), 15(17); | Found in: |
| | | 1(14), 7(8), 15(17) | Nasutitermes exitiosus (first described herein) |
| 5 | trinervitatetraenes | 1(14), 8(19), 9(10), 15(17) | Found in: Nasutitermes exitiosus (first described herein) |

Note:
1(14) and 9(10) are new double bond positions in trinervitane derivatives, 7(8) is new among natural trinervitane derivatives.

Literature Cited:
[1] DeCoursey, J. D.; Webster, A. P.; Taylor, W. W.; Leopold, R. S.; Kathan, R. H. Ann. Entomol. Soc. Am. 1953, 46, 386-392.
[2] Leem, J. Y.; Jeong, I. J.; Park, K. T.; Park, H. Y. FEBS Letts 1999,442, 53-56.
[3] Silverstein, R. M.; Bassler, G. C.; Morrill, T. C. Spectrometric Identification of Organic Compounds; John Wiley & Sons: New York, 1981.
[4] Valterova, I.; Budesinsky, M.; Vrkoc, J. Collection Czechoslov. Chem. Commun. 1991, 56,2969-2977.
[5] Vrkoc, J.; Budesinsky, M.; Sedmera, P. Collection Czechoslov. Chem. Commun. 1978, 43, 1125-1132.
[6] Valterova, I.; Budesinsky, M.; Turecek, F.; Vrkoc, J. Collection Czechoslov. Chem. Commun. 1984, 49, 2024-2039.
[7] Baker, R.; Walnsley, S. Tetrahedron 1982, 38, 1899-1910.
[8] Valterova, I.; Vasickova, S.; Budesinsky, M.; Vrkoc, J. Collection Czechoslov. Chem. Commun. 1986, 51, 2884-2895.
[9] Prestwich, G. D.; Tanis, P.; Springer, J. P.; Clardy, J. J. Am. Chem. Soc. 1976, 98, 6061-6062.
[10] Hirukawa, T.; Suzuki, T.; Tanaka, M.; Kato, T. J. Chem. Soc., Chem. Commun. 1994, 311-312.
[11] Braekman, J. C.; Daloze, D.; Dupont, A.; Pasteels, J. M.; Josens, G. J. Chem. Ecol. 1984, 10, 1363-1370.
[12] Chuah, C. H.; Goh, S. H.; Tho, Y. P. J. Chem. Ecol. 1986, 12,701-712.
[13] Dupont, A.; Braekman, J. C.; Daloze, D.; Pasteels, J. M.; Tursch, B. Bull. Soc. Chim. Belg. 1981,90, 485-499.
[14] Goh, S. H.; Chuah, C. H.; Tho, Y. P.; Prestwich, G. D. J. Chem. Ecol. 1984, 10, 929-944.
[15] Goh, S. H.; Chuah, C. H.; Vadiveloo, J.; Tho, Y. P. J. Chem. Ecol. 1990, 16, 619-630.
[16] Prestwich, G. D. Biochem. Syst. Ecol. 1979, 7,211-221.
[17] Prestwich, G. D. Insect Biochem. 1979, 9, 563-567.
[18] Prestwich, G. D.; Chen, D. J. Chem. Ecol 1981, 7, 147-157.
[19] Vrkoc, J.; Budesinsky, M.; Sedmera, P. Collection Czechoslov. Chem. Commun. 1978, 43,2478-2485.
[20] Goh, S. H.; Tong, S. L.; Tho, Y. P. Mikrochimica Acta 1982, 1, 219-229.
[21] Dauben, W. G.; Lorenz, K. L.; Dean, D. W.; Shapiro, G.; Farkas, I. Tet. Letters 1998, 39, 7079-7082.
[22] Kato, T.; Hirukawa, T.; Suzuki, T.; Tanaka, M.; Hoshikawa, M.; Yagi, M.; Tanaka, M.; Takagi, S. S.; Saito, N. Helv. Chim. Acta 2001, 84, 47-68.
[23] Kato, T.; Tanaka, M.; Hoshikawa, M.; Yagi, M. Tet. Letters 1998, 39, 7553-7556.
[24] Braekman, J. C.; Daloze, D.; Dupont, A.; Pasteels, J. M.; Lefeuve, P.; Bordereau, C.; Declercq, J. P.; Meerssche, M. V. Tetrahedron 1983, 39, 4237-4241.
[25] Chuah, C. H.; Goh, S. H.; Blunt, J. W. Biochem. Syst. Ecol. 1991, 1991, 35-46.
[26] Chuah, C. H.; Goh, S. H.; Beloeil, J. C.; Morellet, N. Malaysian Journal of Science 1987, 9, 83-90.
[27] Chuah, C. H.; Goh, S. H.; Tho, Y. P. J. Chem. Ecol. 1989, 15, 549-563.
[28] Trost, B. M. (Editor-in-Chief) Comprehensive Organic Synthesis. Selectivity, Strategy and Efficiency in Modern Organic Chemistry, Vols 1-9; Pergamon Press: Oxford, 1991.
[29] ApSimon, J. (Editor) The Total Synthesis of Natural Products, Vols.1-9, Wiley and Sons: New York, 1973-1992.

1. A compound of the formula: 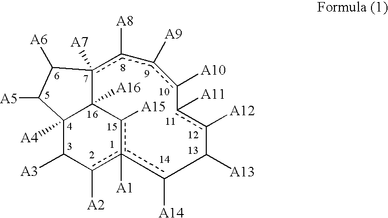
wherein the compound comprises at least one double bond between the carbon atoms selected from the group consisting of: C1 and C14, C9 and C10, and C7 and C8; and
wherein; each [symbol] independently denotes a single or double bond or an epoxidised bond, and
(i) substituents A¹ to A¹⁶ are selected, independently, from H, OH, O, SH, NH₂, lower alkyl, lower alkene, lower alkyne, lower alkoxy, lower carboxy, lower aldehyde groups, lower ketone groups, lower ester groups, lower acyloxy groups, lower alcohol groups, lower alkylthio, lower alkylamino, lower alkylsulfonyl, lower alkylsulfinyl and lower alkylsulfonyloxy, or
(ii) any one or more of substituent pairs A¹ and A², A¹ and A³, A² and A³, A² and A⁴, A³and A⁴, A³ and A⁵, A⁴ and A⁵, A⁴ and A⁶, A⁵ and A⁶, A⁵ and A⁷, A⁶ and A⁷, A⁶ and A⁸, A⁷and A⁸, A⁷ and A⁹, A⁸ and A⁹, A⁸and A¹⁰, A⁹ and A¹⁰, A⁹ and A¹¹, A¹⁰ and A¹¹, A¹⁰ and A¹², A¹¹ and A¹², A¹¹ and A¹³, A¹² and A¹³, A¹² and A¹⁴, A¹³ and A¹⁴, A¹ and A¹³, A¹ and A¹⁴, and A² and A¹⁴ form a substituted or unsubstituted heterocyclic group, wherein any substituents, including A¹⁵ and A¹⁶, not forming a substituted or unsubstituted heterocyclic ring, are selected independently from H, OH, O, SH, NH₂, lower alkyl, lower alkene, lower alkyne, lower alkoxy, lower carboxy, lower aldehyde groups, lower ketone groups, lower ester groups, lower acyloxy groups, lower alcohol groups, lower alkylthio, lower alkylamino, lower alkylsulfonyl, lower alkylsulfinyl and lower alkylsulfonyloxy; and
wherein A¹ or A⁷ may be absent; and
wherein any one of A², A³, A⁵, A⁶ and A⁸ to A¹⁵ may be bonded to the ring structure of Formula (1) by a single or double bond or an epoxidised bond; and
optical isomers, hydrates, solvates and/or salts or derivatives thereof;
with the proviso that the compound is not 7(8),11(12),15(17)-trinervitatriene-2α,3α-diol.

2. The compound of claim 1 which comprises at least one double bond between C1 and C14.

3. The compound of claim 1 which comprises at least one double bond between the carbon atoms selected from the group consisting of: C1 and C14, and C9 and C10.

4. The compound of claim 1 which has at least three double bonds.

5. The compound of claim 1 which comprises double bonds at least between the carbon atoms selected from the groups consisting of;
i) C1 and C14, C9 and C10, and C8 and A⁸,
ii) C1 and C14, C15 and A¹⁵, and C8 and A⁸, or
iii) C1 and C14, C15 and A¹⁵, and C7 and C8.

6. The compound of claim 1, wherein A¹, A², A³, A⁵, A⁶, A⁷, A⁹, A¹⁰, A¹¹, A¹³, A¹⁴ and A¹⁶ are selected, independently, from H, OH, O, SH, NH₂ and OR.

7. The compound of claim 6, wherein the R in the group OR is a lower alkyl or lower acyl.

8. The compound of claim 6, wherein A⁷ and/or A¹⁶ are H.

9. The compound of claim 1, wherein A⁴ and A¹² are selected, independently, from lower alkyl, lower carboxy, lower aldehyde groups, lower ketone groups, lower ester groups, lower acyloxy and lower alcohol groups.

10. The compound of claim 9, wherein A⁴ and A¹² are selected, independently, from methyl, hydroxymethyl, formyl and carboxyl.

11. The compound of claim 10, wherein A⁴ and A¹² are methyl.

12. The compound of claim 1, wherein A⁸ and A¹⁵ are selected from lower alkyl, lower alkene or lower alkyne.

13. The compound of claim 12, wherein A⁸ and A¹⁵ are selected from methyl and CH₂.

14. The compound of claim 13, wherein A⁸ and A¹⁵ are CH₂.

15. The compound of claim 1, wherein at least two of A¹ to A¹⁶ are OH or OR.

16. The compound of claim 15, wherein the R in the group OR is a lower alkyl or lower acyl.

17. The compound of claim 15, wherein at least A² and A³ are OH.

18. The compound of claim 1 which comprises the formula: 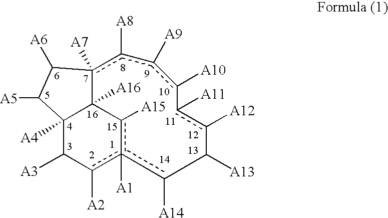
wherein the compound comprises at least one double bond between the carbon atoms selected from the group consisting of: C1 and C14, C9 and C10, and C7 and C8; and
wherein; each [symbol] independently denotes a single or double bond or an epoxidised bond, and
wherein substituents A¹ to A¹⁶ are selected, independently, from H, OH, O, SH, NH₂, lower alkyl, lower alkene, lower alkyne, lower alkoxy, lower carboxy, lower aldehyde groups, lower ketone groups, lower ester groups, lower acyloxy groups, lower alcohol groups, lower alkylthio, lower alkylamino, lower alkylsulfonyl, lower alkylsulfinyl and lower alkylsulfonyloxy, and
wherein A¹ or A⁷ may be absent; and
wherein any one of A², A³, A⁵, A⁶ and A⁸ to A¹⁵ may be bonded to the ring structure of Formula (1) by a single or double bond or an epoxidised bond; and
optical isomers, hydrates, solvates and/or salts ore derivatives thereof;
with the proviso that the compound is not 7(8),11(12),15(17)-trinervitatriene-2α,3α-diol.

19. The compound of claim 1 which comprises the formula: 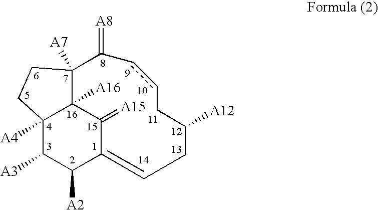
wherein; [symbol] denotes a single or double bond; and
wherein substituents A², A³, A⁴, A⁷, A⁸, A¹², A¹⁵ and A¹⁶ are as defined above in relation to Formula (1), and
optical isomers, hydrates, solvates and/or salts or derivatives thereof.

20. The compound of claim 1 which comprises the formula: 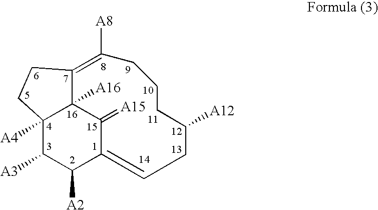
wherein substituents A², A³, A⁴, A⁸, A¹², A¹⁵ and A¹⁶ are as defined above in relation to Formula (1), and
optical isomers, hydrates, solvates and/or salts or derivatives thereof.

21. The compound of claim 1, wherein the salt is a pharmaceutical and/or veterinary acceptable salt.

22. The compound of claim 1, which is selected from the group consisting of: (9E)-1(14),8(19),9(10),15(17)-trinervitatriene-2β,3α-diol (P); 1(14),8(19),15(17)-trinervitatriene-2α,3α-diol (Q); and 1(14),7(8),15(17)-trinervitatriene-2β,3α-diol (R).

23. A pharmaceutical and/or veterinary formulation for treating a microbial infection or disease in a subject, said formulation comprising a compound of claim 1 in admixture with a suitable pharmaceutically/veterinary-acceptable excipient.

24. A composition comprising a compound of claim 1 in admixture with at least one diluent.

25. A method for treating or preventing a microbial infection or disease in a subject, the method comprising administering or applying to the subject an effective amount of a compound of claim 1.

26. (canceled)

27. A method for disinfecting a surface or a solution, said method comprising exposing said surface or solution to an effective amount of a compound of claim 1.

28. A kit comprising a compound claim 1.
